(19) United States
(12) Patent Application Publication
(10) Pub. No.: US 2020/0368059 A1
(43) Pub. Date: Nov. 26, 2020

(54) MENSTRUAL CUP AND METHOD OF USE

(71) Applicant: Jodie Greb, Paris, TX (US)

(72) Inventor: Jodie Greb, Paris, TX (US)

(21) Appl. No.: 16/417,211

(22) Filed: May 20, 2019

(51) Int. Cl. A61F 5/455

(52) U.S. Cl. CPC A61F 5/4553

(57) ABSTRACT

A menstrual cup includes a body extending from a top end to a bottom end, the body forming a tapered receptacle for receiving and collecting fluid; and a ring extending from a first end to a second end, the first end attached to the body and the ring curving underneath the body to attach to the second end to the body, thereby forming an opening; the body and ring are composed of a body safe material.

### BACKGROUND

### 1. Field of the Invention

The present invention relates generally to menstrual cups, and more specifically, to a menstrual cup with a ring for easy removal of the menstrual cup.

### 2. Description of Related Art

Menstrual cups are well known in the art and are effective means to collect menstrual fluid from a woman. For example, FIG. 1 depicts a conventional menstrual cup 101 having a body 103 and a stem 105, wherein the body 103 has an opening 107. During use, the menstrual cup 101 is placed inside a woman's vaginal cavity for collecting menstrual fluid.

One of the problems commonly associated with cup 101 is limited use. For example, the user may find it difficult or uncomfortable to grab the stem for removal. This may cause some women to decide that menstrual cups are not suitable for them or may cause them anxiety during use.

Accordingly, although great strides have been made in the area of menstrual cups, many shortcomings remain.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a front view of a common menstrual cup;

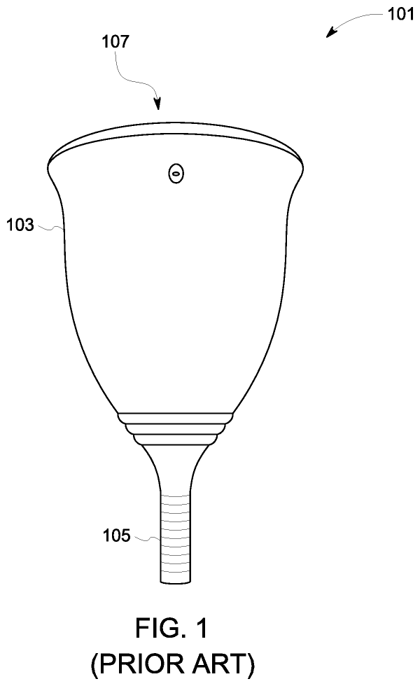

FIG. 2 is an isometric view of a menstrual cup in accordance with a preferred embodiment of the present application; and

FIG. 3 is a flowchart of a method of use of the menstrual cup of FIG. 2.

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional menstrual cups. Specifically, the present invention provides a ring for easy removal of the menstrual cup. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

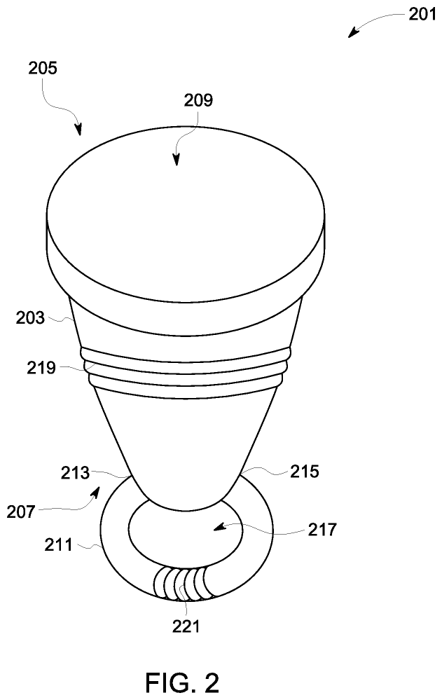

Referring now to the drawings wherein like reference characters identify corresponding or similar elements throughout the several views, FIG. 2 depicts an isometric view of a menstrual cup 201 in accordance with a preferred embodiment of the present application. It will be appreciated that cup 201 overcomes one or more of the above-listed problems commonly associated with conventional menstrual cups.

In the contemplated embodiment, cup 201 includes a body 203 extending from a top end 205 to a bottom end 207 and forming a tapered receptacle 209 for receiving menstrual fluid. In the preferred embodiment, cup 201 further includes a ring 211 extending from a first end 213 to a second end 215 and connecting to the boy 203 to form an opening 217 in the shape of a semi-circle. The opening 217 providing for a user to grip the ring 211 for removal. In some embodiments, the ring 211 is large enough for the user to insert a finger therethrough for easy removal.

It should be appreciated that one of the unique features believed characteristic of the present application is the ring 211, which provides for an easy means of removal of the menstrual cup. It should be appreciated that in the preferred embodiment, the cup 201 does not include a stem at all, as is conventional in the prior art, thereby providing for an improved means of grabbing the ring.

As further shown in FIG. 2, in some embodiments, the cup 201 can include one or more ridges 219 extending around a circumference of the body 203, thereby providing for increased surface tension. In addition, it is contemplated that in some embodiments, the ring 211 can also include ridges 221 for improved grip.

It should be appreciated that the body and ring are both composed of body safe materials, such as a flexible silicone or the like. The color, exact size, and exact materials can vary based on aesthetical, functional, or manufacturing considerations.

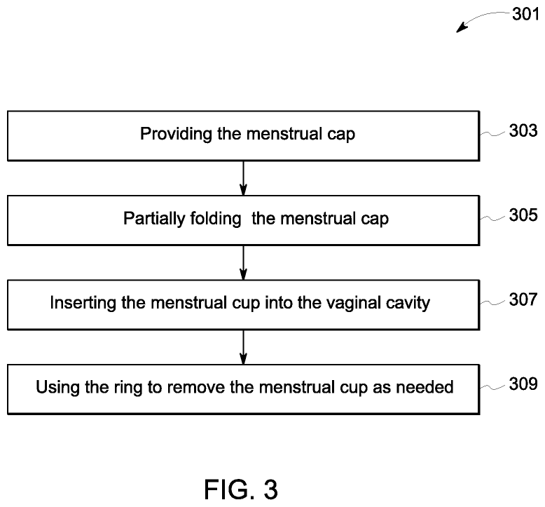

In FIG. 3, a flowchart 301 depicts a method of use of the cup 201. During use, the cup is provided and can be partially folded for insertion into a vaginal cavity, as shown with boxes 303, 305, 307. As desired by the user, the user can grasp the ring and remove the cup, as shown with box 309.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed is:

1. A menstrual cup, comprising:
a body extending from a top end to a bottom end, the body forming a tapered receptacle for receiving and collecting fluid; and
a ring extending from a first end to a second end, the first end attached to the body and the ring curving underneath the body to attach to the second end to the body, thereby forming an opening;
wherein the body and ring are composed of a body safe material.

2. The menstrual cup of claim 1, wherein the body safe material is a silicone.

3. The menstrual cup of claim 1, further comprising:
one or more ridges incorporated into the ring to provide a grip on the ring.

4. The menstrual cup of claim 1, further comprising:
one or more ridges attached to an outer surface of the body.

5. The menstrual cup of claim 1, wherein the ring forms a semi-circle.

6. A method of using a menstrual cup, the method comprising:
providing the menstrual cup of claim 1;
inserting the menstrual cup into a vaginal cavity; and
using the ring to remove the menstrual cup.
